(19) United States
(12) Patent Application Publication
(10) Pub. No.: US 2025/0144374 A1
(43) Pub. Date: May 8, 2025

(54) MEDICAL DEVICE INTRODUCERS, METHODS OF MAKING THE SAME, AND METHODS OF STERILIZATION OF THE SAME

(71) Applicant: EDWARDS LIFESCIENCES CORPORATION, Irvine, CA (US)

(72) Inventors: Sean Chow, Irvine, CA (US); Jeong Soo Lee, Diamond Bar, CA (US); Kevin Jiang, Irvine, CA (US)

(21) Appl. No.: 19/015,139

(22) Filed: Jan. 9, 2025

Related U.S. Application Data
(63) Continuation of application No. PCT/US2023/027490, filed on Jul. 12, 2023.
(60) Provisional application No. 63/369,328, filed on Jul. 25, 2022.

(51) Int. Cl. A61M 25/06; A61M 25/00

(52) U.S. Cl. CPC A61M 25/0662; A61M 25/0009; A61M 25/0023; A61M 25/0045; A61M 25/0068; A61M 25/0097; A61M 2025/0047; A61M 2205/0216; A61M 2205/025; A61M 2207/10

(57) ABSTRACT

Disclosed is an introducer for a medical device wherein the introducer is configured to be substantially sterilized throughout entire d₁ and d₂ along substantially entire I₁ and the introducer includes a first and a second polymer layer that are coextruded.

### CROSS-REFERENCE TO RELATED APPLICATIONS

This application is a continuation of PCT Patent Application No. PCT/US2023/027490 filed Jul. 12, 2023, which claims priority and benefit to U.S. Provisional Application No. 63/369,328, filed Jul. 25, 2022, each of these applications being incorporated herein by reference in its entirety.

### FIELD

The present application concerns aspects of introducers for medical devices, methods of making the same, and methods of sterilization of the same.

### BACKGROUND

The human heart can suffer from various valvular diseases. These valvular diseases can result in significant malfunctioning of the heart and ultimately require the replacement of the native valve with an artificial valve. There are a number of known artificial valves and a number of known methods of implanting these artificial valves in humans.

When the native valve is replaced, surgical implantation of the prosthetic valve typically requires an open-chest surgery during which the heart is stopped, and the patient is placed on cardiopulmonary bypass (a so-called “heart-lung machine”). In one common surgical procedure, the diseased native valve leaflets are excised, and a prosthetic valve is sutured to the surrounding tissue at the valve annulus. Because of the trauma associated with the procedure and the attendant duration of extracorporeal blood circulation, some patients do not survive the surgical procedure or die shortly thereafter. It is well known that the risk to the patient increases with the amount of time required for extracorporeal circulation. Due to these risks, a substantial number of patients with defective native valves are deemed inoperable because their condition is too frail to withstand the procedure. By some estimates, more than 50% of the subjects suffering from valve stenosis who are older than 80 years cannot be operated on for valve replacement.

Because of the drawbacks associated with conventional open-heart surgery, percutaneous and minimally-invasive surgical approaches are garnering intense attention. In one technique, a prosthetic valve is configured to be implanted in a much less invasive procedure by way of catheterization. For instance, U.S. Pat. Nos. 5,411,522 and 6,730,118, which are incorporated herein by reference, describe collapsible transcatheter heart valves that can be percutaneously introduced in a compressed state on a catheter and expanded in the desired position by balloon inflation or by utilization of a self-expanding frame or stent.

To deliver the valve to the implantation location, specifically designed delivery systems are utilized. One of the important components of such a system is a medical device introducer. The introducer is configured to help deliver the prosthetic valve and to remove a guide wire from the implantation site. To easily, with a minimal invasion, deliver the valve, the introducer often has a length that exceeds 30 inches. However, to prevent air emboli, it is desired to keep the inner diameter of the introducer very small (less than about 0.05 inches) along the entire length of the device. However, such a high aspect-ratio device is difficult to sterilize efficiently. To ensure the desired level of sterilization, current introducers are built to have an inner diameter of about 0.150 inches along most of the length of the introducer and only have an inner diameter of less than about 0.05 inches in the distal end of the device. However, such constructions are less preferable as air emboli remain a substantial threat to patient health.

Therefore, there is a need for new introducers and methods of making the same, as well as methods of sterilization of the same introducers that substantially eliminate these issues and improve the safety of the patient. These needs and other needs are at least partially satisfied by the present disclosure.

### SUMMARY

Aspects of the present disclosure relate to prosthetic valves. Some aspects relate to an introducer for a medical device comprising: an elongated tubing that comprises a first polymer layer forming an outer surface of the introducer having an outer diameter d₁ and a second polymer layer forming an inner surface of the introducer and defining a lumen having a diameter d₂. In some implementations, the second layer is substantially more hydrophobic than the first layer. In some implementations, the elongated tubing has a length I₁ and a ratio of I₁/d₁ is greater than about 120, and a ratio of I₁/d₂ is greater than about 200 to about 2,000. In yet some implementations, the introducer is configured to be substantially sterilized throughout of substantially entire d₁ and d₂ along substantially entire I₁.

Also disclosed is the introducer for the medical device of any disclosed herein aspects, wherein the second polymer layer comprises one or more polypropylene, polyethylene, a graft-modified polyethylene, or a graft-modified polypropylene.

Also disclosed is the introducer for the medical device of any disclosed herein aspects, wherein the second polymer layer comprises one or more of a low-density polyethylene (LDPE), medium density polyethylene, ultra-low-density polyethylene (ULDPE), high density polyethylene (HDPE), heterogeneously branched linear low-density polyethylene (LLDPE), a graft modified ethylene polymer, grafted modified polypropylene, or ethylene-vinyl acetate (EVA), or any combination thereof.

Also disclosed is the introducer for the medical device of any disclosed herein aspects, wherein the graft-modified polyethylene or graft-modified polypropylene is maleic anhydride graft-modified or acrylic acid graft-modified.

In some implementations, also disclosed is the introducer for the medical device of any disclosed herein aspects, wherein the first polymer layer comprises polyurethane, polyether block amide, polyethylene oxide, or any combination thereof.

In some implementations, also disclosed is the introducer for the medical device of any disclosed herein aspects, wherein the first polymer layer is a thermoplastic polyurethane.

In some implementations, also disclosed is the introducer for the medical device of any disclosed herein aspects, wherein the first polymer layer is a compound comprising greater than 0 wt % to less than 100 wt % of a polyether-based thermoplastic polyurethane and about 5 wt % to about 50 wt % of polyethylene oxide.

In some implementations, also disclosed is the introducer for the medical device of any disclosed herein aspects, wherein the polyurethane has a durometer Shore D of about 50D to about 75D.

In some implementations, also disclosed is the introducer for the medical device of any disclosed herein aspects, wherein the polyurethane has a Shore A of about 25A to about 95A.

In some implementations, also disclosed is the introducer for the medical device of any disclosed herein aspects, wherein the first polymer layer is a polyether block amide.

In some implementations, also disclosed is the introducer for the medical device of any disclosed herein aspects, wherein the first polymer layer is a compound comprising greater than 0 wt % to less than 100 wt % of a polyether block amide and about 5 wt % to about 50 wt % of polyethylene oxide.

In some implementations, also disclosed is the introducer for the medical device of any disclosed herein aspects, wherein the polyether block amide has a Shore hardness of about 25 D to about 72 D.

In some implementations, also disclosed is the introducer for the medical device of any disclosed herein aspects, wherein the polyethylene oxide (PEO) has a molecular weight of about 100,000 to about 7,000,000.

In some implementations, also disclosed is the introducer for the medical device of any disclosed herein aspects, wherein the first polymer layer has a thickness of about 0.005 inches to about 0.150 inches.

In some implementations, also disclosed is the introducer for the medical device of any disclosed herein aspects, wherein the second polymer layer has a thickness of about 0.005 inches to about 0.035 inches.

In some implementations, also disclosed is the introducer for the medical device of any disclosed herein aspects, wherein comprises a third polymer layer positioned between the first and the second polymer layers.

In some implementations, also disclosed is the introducer for the medical device of any disclosed herein aspects, wherein the third polymer layer is a tie layer and comprises a graft-modified polyethylene or graft-modified polypropylene.

In some implementations, also disclosed is the introducer for the medical device of any disclosed herein aspects, wherein the third polymer layer has a thickness of about 0.001 inches to about 0.025 inches.

In some implementations, also disclosed is the introducer for the medical device of any disclosed herein aspects, wherein the first polymer layer and/or the second polymer layer comprise a filler.

In some implementations, also disclosed is the introducer for the medical device of any disclosed herein aspects, wherein the third polymer layer comprises a filler.

In some implementations, also disclosed is the introducer for the medical device of any disclosed herein aspects, wherein the filler comprises bismuth oxychloride, barium sulfate, bismuth subcarbonate, calcium carbonate, aluminum trihydrate, barite, kaolin clay, limestone, titanium oxide, or any combination thereof.

In some implementations, also disclosed is the introducer for the medical device of any disclosed herein aspects, wherein the first and the second polymer layers are coextruded.

In some implementations, also disclosed is the introducer for the medical device of any disclosed herein aspects, wherein the first polymer layer and/or the second polymer layer and/or third layer are co-extruded.

In some implementations, also disclosed is the introducer for the medical device of any disclosed herein aspects, wherein the first polymer layer and/or the second polymer layer are at least partially crosslinked.

In some implementations, also disclosed is the introducer for the medical device of any disclosed herein aspects, wherein the crosslinking is achieved by exposure to an energy source, wherein the energy source is selected from e-beam radiation, X-ray radiation, gamma-radiation, or electromagnetic radiation.

In some implementations, also disclosed is the introducer for the medical device of any disclosed herein aspects, wherein the crosslinking is achieved by exposure to the energy source at a dose of about 25 kGrey to about 100 kGrey.

In some implementations, also disclosed is the introducer for the medical device of any disclosed herein aspects, wherein the first polymer layer and/or the second polymer layer are substantially crosslinked.

In some implementations, also disclosed is the introducer for the medical device of any disclosed herein aspects, wherein the inner diameter d₂ is substantially the same along the entire length I₁.

In some implementations, also disclosed is the introducer for the medical device of any disclosed herein aspects, wherein the introducer has a proximal portion and a distal portion.

In some implementations, also disclosed is the introducer for the medical device of any disclosed herein aspects, wherein the proximal portion is coupled with a hub or an adaptor to connect a medical device.

In some implementations, also disclosed is the introducer for the medical device of any disclosed herein aspects, wherein the distal portion is tapered such that the outer diameter d₁ decreases to a distal end of the introducer while the inner diameter d₁ remains substantially the same along the entire length I₁.

In some implementations, also disclosed is the introducer for the medical device of any disclosed herein aspects, wherein the introducer is configured to receive a guidewire used to deploy the medical device.

In some implementations, also disclosed is the introducer for the medical device of any disclosed herein aspects, wherein the sterilization is ethylene oxide-based.

In some implementations, also disclosed is the introducer for the medical device of any disclosed herein aspects, wherein substantially sterilized throughout of substantially entire d₂ along substantially entire I₁ defined by achieving effective inactivation of bio-indicators in a fractional cycle to less than about 1 CFU.

In some implementations, also disclosed is the introducer for the medical device of any disclosed herein aspects, wherein a sterility assurance level is about 10-6.

In some implementations, also disclosed is the introducer for the medical device of any disclosed herein aspects, wherein the outer surface of the introducer is substantially lubricious and wherein exhibits a Coefficient of Friction (COF) of about 0.05 to about 0.5.

In some implementations, also disclosed is the introducer for the medical device of any disclosed herein aspects, wherein the outer surface of the introducer does not comprise a hydrophilic coating.

In some implementations, also disclosed is the introducer for the medical device of any disclosed herein aspects, wherein the introducer is configured to generate substantially no insoluble particulate material.

In some implementations, disclosed is a delivery system comprising: the introducer for the medical device of any examples herein, particularly examples 1-38; a delivery sheath; and a prosthetic valve.

In some implementations, disclosed is a method of making the introducer for the medical device of any examples herein, particularly examples 1-38 comprising: forming an elongated tubing comprising a first polymer layer forming an outer surface of the introducer having an outer diameter d₁ and a second polymer layer; forming an inner surface of the introducer and defining a lumen having a diameter d₂, wherein the second layer is substantially more hydrophobic than the first layer; wherein the elongated tubing has a length I₁, wherein a ratio of I₁/d₁ is greater than about 120 and a ratio of I₁/d₂ is greater than about 200 to about 2,000; and wherein the introducer is configured to be substantially sterilized throughout of substantially entire d₁ and d₂ along substantially entire I₁.

In some implementations, disclosed is the method of any disclosed herein aspects, wherein the step of forming comprises co-extruding the first and the second polymer layers.

In some implementations, disclosed is the method of any disclosed herein aspects, wherein the elongated tubing further comprises a third polymer layer positioned between the first and the second polymer layers.

In some implementations, disclosed is the method of any disclosed herein aspects, wherein the third polymer layer is coextruded with the first and the second polymer layers.

In some implementations, disclosed is a method of delivering a prosthetic valve comprising: placing a guide wire; introducing the introducer of any one of examples herein, particularly examples 1-38, removing the guide wire; and placing a prosthetic valve in an implantation site.

In some implementations, disclosed is a method comprising: exposing the introducer for the medical device any examples herein, particularly examples 1-38, to ethylene oxide gas comprising about 20% to about 70% of humidity for a predetermined time at a predetermined temperature.

In some implementations, disclosed is the method of any disclosed herein aspects, wherein the step of exposing comprises one or more cycles of exposing to the ethylene oxide gas.

In some implementations, disclosed is the method of any disclosed herein aspects, wherein the step of exposing achieves effective inactivation of bio-indicators in a fractional cycle to less than about 1 CFU.

In some implementations, disclosed is the method of any disclosed herein aspects, wherein the step of exposing achieves a sterility assurance level is about 10-6.

In some implementations, disclosed is the method of any disclosed herein aspects, wherein the first polymer layer, the second polymer layer, and the third polymer layer show substantially no deterioration after the step of exposing.

The above method(s) can be performed on a patient or a living animal or on a simulation, such as on a cadaver, cadaver heart, anthropomorphic ghost, or simulator (e.g., with body parts, heart, tissue, etc. being simulated).

“Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).”

The foregoing and other features and advantages of the disclosure will become more apparent from the following detailed description, which proceeds with reference to the accompanying figures.

### BRIEF DESCRIPTION OF THE DRAWINGS

FIGS. 1A-1B show a schematic of an exemplary introducer in one aspect.

FIG. 1A shows a schematic of an entire introducer. FIG. 1B shows a schematic of a tapered portion of the introducer in one aspect.

FIG. 2 shows a schematic of various layers of the introducer in one aspect.

FIG. 3 shows a schematic of various layers of the introducer in one aspect in a different aspect.

FIGS. 4A-4B show an exemplary introducer and delivery system. FIG. 4A shows the introduction of the introducer into a delivery sheath. FIG. 4B shows a general representation of the delivery system with the introducer.

FIG. 5 shows a schematic of one of the steps of implantation procedures in one aspect.

### DETAILED DESCRIPTION

The present disclosure can be understood more readily by reference to the following detailed description, examples, drawings, and claims, and their previous and following description. However, before the present articles, systems, and/or methods are disclosed and described, it is to be understood that this disclosure is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

Those skilled in the relevant art will recognize and appreciate that many changes can be made to the various aspects of the disclosure described herein while still obtaining the beneficial results of the present disclosure. It will also be apparent that some of the desired benefits of the present disclosure can be obtained by selecting some of the features of the present disclosure without utilizing other features. Accordingly, those of ordinary skill in the pertinent art will recognize that many modifications and adaptations to the present disclosure are possible and may even be desirable in certain circumstances and are a part of the present disclosure. Thus, the following description is again provided as illustrative of the principles of the present disclosure and not in limitation thereof.

### Definitions

As used in this application and in the claims, the singular forms “a,” “an,” and “the” include the plural forms unless the context clearly dictates otherwise. Thus, for example, reference to an “introducer” includes aspects having two or more such introducers unless the context clearly indicates otherwise.

It is also to be understood that the terminology used herein is for the purpose of describing particular aspects only and is not intended to be limiting. As used in the specification and in the claims, the term “comprising” can include the aspects “consisting of” and “consisting essentially of.” Additionally, the term “includes” means “comprises.”

For the terms “for example” and “such as” and grammatical equivalences thereof, the phrase “and without limitation” is understood to follow unless explicitly stated otherwise. It is further understood that these phrases are used for explanatory purposes only. It is further understood that the term “exemplary,” as used herein, means “an example of” and is not intended to convey an indication of a preferred or ideal aspect.

The term “or” means “and/or.” Recitation of ranges of values is merely intended to serve as a shorthand method of referring individually to each separate value falling within the range unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context.

Ranges can be expressed herein as from “about” one particular value and/or to “about” another particular value. When such a range is expressed, some implementation includes from the one particular value and/or to the other particular value. Similarly, when values are expressed as approximations, by use of the antecedent “about,” it will be understood that the particular value forms another aspect. It should be further understood that the endpoints of each of the ranges are significant both in relation to the other endpoint and independently of the other endpoint.

Recitation of ranges of values herein is merely intended to serve as a shorthand method of referring individually to each separate value recited or falling within the range unless otherwise indicated herein, and each separate value is incorporated into the specification as if it were individually recited. Ranges provided herein are understood to be shorthand for all of the values within the range. For example, a range of 1 to 50 is understood to include any number, or combination of numbers, from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or sub-ranges from the group consisting of 10-40, 20-50, 5-35, etc. Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4).

Further, the terms “coupled” and “associated” generally mean electrically, electromagnetically, and/or physically (e.g., mechanically or chemically) coupled or linked and do not exclude the presence of intermediate elements between the coupled or associated items.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation.

As used herein, the term “substantially” means that the subsequently described event or circumstance completely occurs or that the subsequently described event or circumstance generally, typically, or approximately occurs.

Still further, the term “substantially” can, in some aspects, refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

In some implementations, as used herein, the term “substantially free,” when used in the context of a composition or component of a composition that is substantially absent, is intended to refer to an amount that is then about 1% by weight, e.g., less than about 0.5% by weight, less than about 0.1% by weight, less than about 0.05% by weight, or less than about 0.01% by weight of the stated material, based on the total weight of the composition.

While aspects of the present disclosure can be described and claimed in a particular statutory class, such as the system statutory class, this is for convenience only, and one of ordinary skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

Although the operations of exemplary aspects of the disclosed methods may be described in a particular sequential order for convenient presentation, it should be understood that disclosed aspects can encompass an order of operations other than the particular sequential order disclosed. For example, operations described sequentially may, in some cases, be rearranged or performed concurrently. Further, descriptions and disclosures provided in association with one particular aspect are not limited to that aspect and may be applied to any aspect disclosed.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

The treatment techniques, methods, steps, etc. described or suggested herein or in references incorporated herein can be performed on a patient, living animal, or on a non-living simulation, such as on a cadaver, cadaver heart, anthropomorphic ghost, simulator (e.g., with the body parts, tissue, etc. being simulated), etc.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The present disclosure may be understood more readily by reference to the following detailed description of various aspects of the invention and the examples included therein and to the Figures and their previous and following description.

### Introducer

In one aspect disclosed herein is an introducer for a medical device. The medical device may comprise an elongated tubing having a length I₁. The tubing may comprise a first polymer layer forming an outer surface of the introducer having an outer diameter d₁ and a second polymer layer forming an inner surface of the introducer and defining a lumen having a diameter d₂. In some aspects, the second layer is substantially more hydrophobic than the first layer. In some aspects, the length I₁ can be anywhere about 30 inches to about 50 inches, including exemplary values of about 31 inches, about 32 inches, about 33 inches, about 34 inches, about 35 inches, about 36 inches, about 37 inches, about 38 inches, about 39 Inches, about 40 inches, about 41 inches, about 42 inches, about 43 inches, about 44 inches, about 45 inches, about 46 inches, about 47 inches, about 48 inches, and about 49 inches. It is understood that the introducers disclosed herein can also be longer than 50 inches.

In some implementations, the diameter d₁ can be about 0.150 inches to about 0.5 inches, including exemplary values of about 0.155 inches, about 0.200 inches, about 0.225 inches, about 250 inches, about 0.275 inches, about 0.300 inches, about 0.325 inches, about 0.350 inches, about 0.375 inches, about 0.400 inches, about 0.425 Inches, about 0.450 inches, and about 0.475 inches.

In some implementations, da can be about 0.01 inches to about 0.07 inches, including exemplary values of about 0.0125 inches, about 0.015 inches, about 0.0175 inches, about 0.020 inches, about 0.0225 inches, about 0.0250 inches, about 0.0275 inches, about 0.030 inches, about 0.0325 inches, about 0.0350 inches, about 0.0375 inches, about 0.040 inches, about 0.0425 inches, about 0.0450 inches, about 0.0475 Inches, about 0.05 inches, about 0.0525 inches, about 0.0550 inches, about 0.0575 inches, about 0.06 inches, about 0.0625 inches, about 0.0650 inches, and about 0.0675 inches.

In some implementations, a ratio between hi and d₁ (I₁/d₁) is greater than about 120, greater than about 130, greater than about 140, greater than about 150, greater than about 160, greater than about 170, greater than about 180, greater than about 190, greater than about 200, greater than about 210, greater than about 220, greater than about 230, greater than about 240, greater than about 250, greater than about 260, greater than about 270, greater than about 280, greater than about 290, greater than about 300, greater than about 310, greater than about 320, greater than about 330, greater than about 340, greater than about 350, greater than about 360, greater than about 370, greater than about 380, greater than about 390, greater than about 400, greater than about 410, greater than about 420, greater than about 430, greater than about 440, greater than about 450, greater than about 460, greater than about 470, greater than about 480, greater than about 490, or even greater than about 500. In some implementations, a ratio between h and d₁ (h/d₁) can be about 100 and about 500, including exemplary values of about 120, about 150, about 180, about 200, about 220, about 250, about 280, about 300, about 320, about 350, about 380, about 300, about 320, about 350, about 380, about 400, about 420, about 450, and about 480.

In some implementations, a ratio of I₁/d₂ is greater than about 200 to about 2,000, including exemplary values of about 250, about 300, about 350, about 400, about 500, about 600, about 700, about 800, about 900, about 1,000, about 1,100, about 1,200, about 1,300, about 1,400, about 1,500, about 1,600, about 1,700, about 1,800, and about 1,900.

One of the issues in the medical arts is the ability to efficiently sterilize high aspect ratio devices. Commonly, an ethylene oxide gas is used to sterilize medical devices. Most medical devices are hygroscopic and can quickly absorb moisture from the ethylene oxide gas mixture during sterilization procedures. As a result, sterilization becomes inefficient. More specifically, the efficient killing of microorganisms in very narrow lumens over a large length of the device becomes problematic. As a result, the sterilization is not uniform and unsatisfactory for surgical procedures as it can lead to risk of the patient's health during operation.

One approach for the longer devices is to increase the inner diameter to the highest acceptable value and only have a narrow inner diameter over a short portion of the device as needed, for example, over a distal end of the device or a proximal end of the device. However, this approach is not preferable. For example, undesirable side effects such as air emboli can still occur in devices with larger diameters.

The introducer disclosed herein overcomes challenges with the sterilization process and is configured to be substantially sterilized throughout substantially entire d₁ and d₂ along substantially entire I₁.

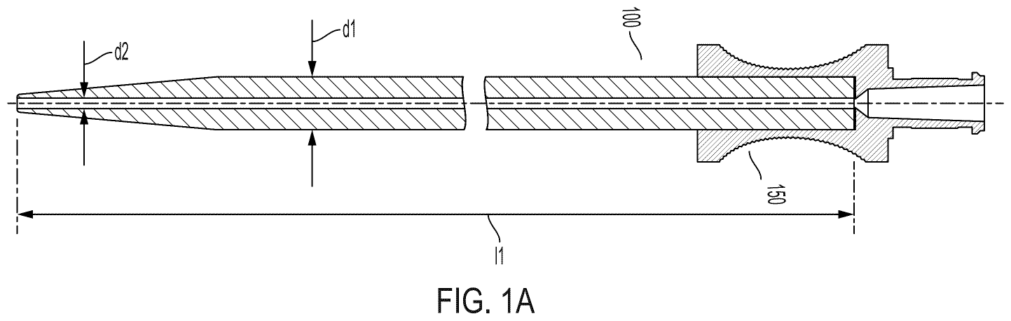

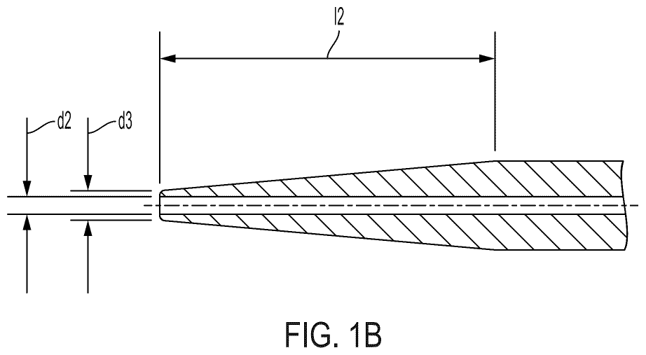

An exemplary introducer 100 for medical devices is shown in FIG. 1A. The introducer has any of the disclosed above I₁, outer diameter d₁, and inner diameter d₂. In some implementations, the inner diameter d₂ can be substantially the same along the entire I₁. While in some implementations, the inner diameter d₂ can vary along the length I₁ depending on the specific application. However, it is understood that the overall value of d₂ falls within the disclosed above range.

In some implementations, the outer diameter d₁ of the introducer can be substantially the same over the entire length I₁, while in some implementations, the diameter d₁ can vary depending on the specific application. As shown in FIG. 1B, at least a portion of the disclosed introducer may be tapered.

It is understood that an introducer disclosed herein has a proximal portion and a distal portion. In some aspects, the distal portion of the introducer can be tapered at length I₂. A such tapering portion can allow a more atraumatic use of the introducer during surgical procedures. In certain aspects, I₂ can be about 0.1 inches to about 3 inches, including exemplary values of about 0.2 inches, about 0.5 inches, about 0.8 inches, about 1.0 inches, about 1.2 inches, about 1.5 inches, about 1.8 inches, about 2.0 inches, about 2.2 inches, about 2.5 inches, and about 2.8 inches extending from a distal end of the introducer towards a proximal end of the introducer. In such aspects, an outer diameter d₃ at the distal end of the tapered portion is substantially smaller than d₁. Yet, in some implementations, an outer diameter d₃ at the distal end of the tapered portion is only incrementally larger than d₂. For example, d₃ can be about 0.05 inches to about 0.3 inches, including exemplary values of about 0.055 inches, about 0.065 inches, about 0.07 inches, about 0.075 inches, about 0.080 inches, about 0.085 inches, about 0.090 inches, about 0.095 inches, about 0.10 inches, about 0.15 inches, about 0.20 inches, and about 0.25 inches.

In some implementations, the distal portion is tapered such that the outer diameter d₁ decreases to a distal end of the introducer while the inner diameter d₁ remains substantially the same along the entire length I₁.

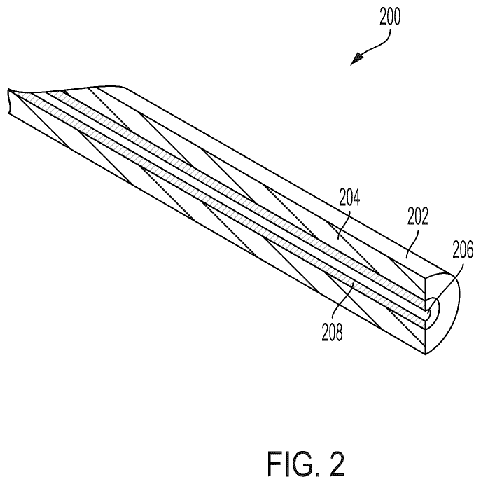

In some implementations, the disclosed herein introducer 200 can comprise at least two layers, as shown in FIG. 2, a first polymer layer 204 that forms an outside surface of the introducer 202 and a second polymer layer 208 that forms a lumen 206 (also defines an inner surface of the introducer).

It is understood that in the disclosed herein aspects, the second polymer layer is more hydrophobic than the first polymer layer. Such a configuration allows the ethylene oxide gas to penetrate and sterilize the inner lumen of the introducer without being immediately absorbed by the polymer layer.

In some aspects, the second polymer layer can comprise one or more polypropylene, polyethylene, graft-modified polyethylene, or graft-modified polypropylene. Yet, in some implementations, the second polymer layer can comprise one or more of low-density polyethylene (LDPE), medium density polyethylene, ultra-low-density polyethylene (ULDPE), high density polyethylene (HDPE), heterogeneously branched linear low-density polyethylene (LLDPE), a graft modified ethylene polymer, grafted modified polypropylene, or ethylene-vinyl acetate (EVA), or any combination thereof. In some implementations, the second polymer layer can comprise LLDPE. While in some implementations, the second polymer layer can comprise HDPE or LDPE.

In some implementations, the graft-modified polyethylene or graft-modified polypropylene is maleic anhydride graft-modified or acrylic acid graft-modified. For example, a graft-modified polyolefin sold under the trademark name of Orevac can be used. It is also understood that the second polymer layer can comprise a copolymer of any of the disclosed above polymers. Yet, in some implementations, the second polymer can be compounded from two or more polymers selected from any of the polymers disclosed above.

In some implementations, the second polymer layer 208 can have a thickness of about 0.005 inches to about 0.035 inches, including exemplary values of about 0.006 inches, about 0.007 inches, about 0.008 inches, about 0.009 inches, about 0.01 inches, about 0.012 inches, about 0.015 inches, about 0.017 inches, about 0.02 inches, about 0.022 inches, about 0.025 inches, about 0.027 inches, about 0.030 inches, and about 0.032 inches.

In some implementations, the first polymer layer can comprise polyurethane, polyether block amide, polyethylene oxide, or any combination thereof. In some aspects, the first polymer layer can be a thermoplastic polyurethane. While in some implementations, the first polymer layer can be a compound material comprising a polyurethane and polyether block amide. In some implementations, the first polymer layer can be a compound material comprising a polyurethane and polyethylene oxide. In yet some implementations, the first polymer layer can be a compound material comprising a polyether block amide and polyethylene oxide. In yet some implementations, the first polymer layer can be a compound material comprising a polyurethane, polyether block amide, and polyethylene oxide.

Yet, in some implementations, the first polymer layer can be a compound comprising greater than 0 wt % to less than 100 wt % of a polyether-based thermoplastic polyurethane and about 5 wt % to about 50 wt % of polyethylene oxide.

In such aspects, the polyether-based thermoplastic can be present in an amount greater than 0 wt % to less than 100 wt %, including exemplary values of about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, and about 95 wt %. The polyether-based thermoplastic polyurethane can be present in any amount between any two foregoing values. In some implementations, the polyether-based thermoplastic polyurethane can be based on an aromatic polyether or aliphatic polyether. In some aspects, the polyether-based thermoplastic polyurethane used in this disclosure can be the one sold under the trademark name of Tecothane™ or TecoFlex™ In some implementations, the polyurethane disclosed herein can include Pellethane® or Texin®.

In some implementations, the polyethylene oxide can be present in an amount of about 5 wt % to about 80 wt %, including exemplary values of about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %. The polyethylene oxide can be present in any amount between any two foregoing values.

In some implementations, the polyurethane disclosed herein can have a durometer Shore D of about 50D to about 75D, including exemplary values of about 55D, about 60D, or about 70D. In some implementations, the polyurethane disclosed herein can have a Shore A of about 25A to about 95A, including exemplary values of about 30A, about 35A, about 40A, about 45A, about 50A, about 55A, about 60A, about 65A, about 70A, about 75A, about 80A, about 85A, and about 90A.

Yet, in some implementations, the first polymer layer is a polyether block amide. In certain aspects, the polyether block amide can be PEBAX. In yet some implementations, other polyamides such as Vestamid® and Nylon® can also be utilized.

In some implementations, the first polymer layer can be a compound comprising greater than 0 wt % to less than 100 wt % of a polyether block amide and about 5 wt % to about 50 wt % of polyethylene oxide.

In such exemplary aspects, the polyether block amide can be present and amount greater than 0 wt % to less than 100 wt %, including exemplary values of about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, and about 95 wt %. The polyether block amide can be present in any amount between any two foregoing values.

In some implementations, the polyethylene oxide can be present in an amount of about 5 wt % to about 80 wt %, including exemplary values of about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %. The polyethylene oxide can be present in any amount between any two foregoing values.

In some implementations, the polyether block amide has a Shore hardness of about 25 D to about 72 D, including exemplary values of about 30 D, about 35 D, about 40 D, about 45 D, about 50 D, about 55 D, about 60 D, about 65 D, and about 70 D.

In some implementations, the polyethylene oxide (PEO) that can be used in the disclosed herein first polymer layer can have a molecular weight of about 100,000 Daltons to about 7,000,000 Daltons, including exemplary values of about 200,000 Daltons, about 300,000 Daltons, about 400,000 Daltons, about 500,000 Daltons, about 600,000 Daltons, about 700,000 Daltons, about 800,000 Daltons, about 900,000 Daltons, about 1,000,000 Daltons, about 1,250,000 Daltons, about 1,500,000 Daltons, about 1,750,000 Daltons, about 2,000,000 Daltons, about 2,250,000 Daltons, about 2,500,000 Daltons, about 2,750,000 Daltons, about 3,000,000 Daltons, about 3,250,000 Daltons, about 3,500,000 Daltons, about 3,750,000 Daltons, about 4,000,000 Daltons, about 4,250,000 Daltons, about 4,500,000 Daltons, about 4,750,000 Daltons, about 5,000,000 Daltons, about 5,250,000 Daltons, about 5,500,000 Daltons, about 5,750,000 Daltons, about 6,000,000 Daltons, about 6,250,000 Daltons, about 6,500,000 Daltons, and about 6,750,000 Daltons.

In some implementations, the first polymer layer has a thickness of about 0.005 inches to about 0.150 inches, including exemplary values of about 0.0075 inches, about 0.01 inches, about 0.05 inches, about 0.075 inches, about 0.100 inches, about 0.105 inches, about 0.110 inches, about 0.115 inches, about 0.120 inches, about 0.125 inches, about 0.130 inches, about 0.135 inches, about 0.140 inches, and about 0.145 inches.

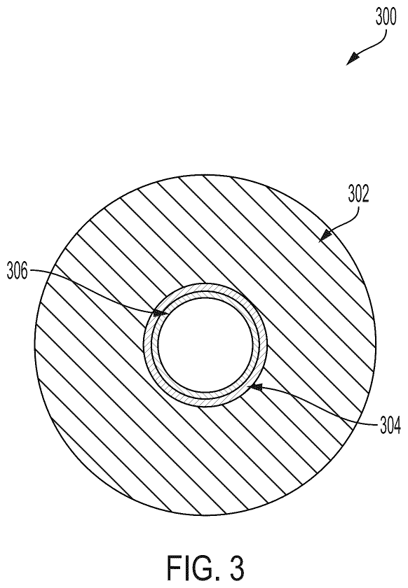

In some implementations, the introducer 300 can comprise the disclosed herein first polymer layer 302, the disclosed herein second polymer layer 306, and an optional third polymer layer 304, as shown in FIG. 3. In such aspects, the third polymer layer 304 can be disposed between the first polymer layer 302 and the second polymer layer 306. In some implementations, the third layer can serve as a tie layer that can bond the first and second polymer layers together. In certain aspects, the third polymer layer can comprise any polymers disclosed herein, or it can comprise any polymers that are configured to serve as a tie layer. In some implementations, the third polymer layer can comprise a graft-modified polyethylene or graft-modified polypropylene. In some implementations, the third polymer layer can comprise Orevac®.

In the aspects where the third polymer layer is present, this polymer layer can have a thickness of about 0.001 inches to about 0.025 inches, including exemplary values of about 0.0015 inches, about 0.002 inches, about 0.0025 inches, about 0.003 inches, about 0.0035 inches, about 0.004 inches, about 0.0045 inches, about 0.005 inches, about 0.0055 inches, about 0.006 inches, about 0.0065 inches, about 0.007 inches, about 0.0075 inches, about 0.008 inches, about 0.0085 inches, about 0.009 inches, about 0.0095 inches, about 0.01 inches, about 0.011 inches, about 0.012 inches, about 0.013 inches, about 0.014 inches, about 0.015 inches, about 0.016 inches, about 0.017 inches, about 0.018 inches, about 0.019 inches, about 0.02 inches, about 0.021 inches, about 0.022 inches, about 0.023 inches, and about 0.024 inches.

In some implementations, the first polymer layer and/or the second polymer layer can comprise a filler. Yet, in some implementations, if the third polymer layer is present, it can also comprise a filler. While in some implementations, if the third polymer layer is present, this layer is substantially free of the filler.

It is understood that the filler, if present in the first polymer layer and/or the second polymer layer and/or third polymer layer, can be the same or different. In some implementations, the filler can comprise bismuth oxychloride, barium sulfate, bismuth subcarbonate, calcium carbonate, aluminum trihydrate, barite, kaolin clay, limestone, titanium oxide, or any combination thereof. In some implementations, the filler can comprise a radiopaque material.

In some implementations, the first and the second layer (and the third layer if present) can be formed by any known in the art methods. For example, each layer can be extruded separately and then assembled together. Yet, in some implementations, the first and the second polymer layers can be co-extruded. In some implementations, if the third polymer layer is present, it can be co-extruded together with the first and the second polymer layer. In yet some implementations, if the third polymer layer is present, it can be coextruded with the first polymer layer and then assembled with the separately extruded second polymer layer. Yet, in some implementations, if the third polymer layer is present, it can be coextruded with the second polymer layer and then assembled with the separately extruded first polymer layer. It is understood that these aspects are exemplary, and any of the disclosed above methods can be used to form the introducer.

In yet some implementations, each or all of the polymer layers present in the introducer can be at least partially crosslinked. For example, the first polymer layer comprising any of the disclosed above polymers or compounds can be at least partially crosslinked. Similarly, the second polymer layer (and/or even the third polymer layer) comprising any of the disclosed above polymers or compounds can be at least partially crosslinked. In some implementations, the first polymer layer can be substantially crosslinked. In some implementations, the second polymer layer (and/or third polymer layer) can be substantially crosslinked.

In some implementations, the crosslinking can be achieved by exposing any or all of the layers to an energy source, wherein the energy source can be selected from e-beam radiation, X-ray radiation, gamma-radiation, or electromagnetic radiation. In certain aspects, the crosslinking is achieved by exposure to the energy source at a dose of about 25 kGrey to about 100 kGrey, including exemplary values of about 30 kGrey, about 35 kGrey, about 40 kGrey, about 45 kGrey, about 50 kGrey, about 55 kGrey, about 60 kGrey, about 65 kGrey, about 70 kGrey, about 75 kGrey, about 80 kGrey, about 85 kGrey, about 90 kGrey, and about 95 kGrey.

In some implementations, where the compounds comprising polyurethane and PEO or the compounds comprising polyether block amide and PEO are used to form the outer surface of the introducer, this outer surface of the introducer is substantially lubricious and wherein exhibits a Coefficient of Friction (COF) of about 0.05 to about 0.5, including exemplary values of about 0.075, about 0.1, about 0.15, about 0.2, about 0.25, about 0.3, about 0.35, about 0.4, and 0.45. It is understood that this coefficient of friction can be measured by any known in the art method. In some exemplary and unlimiting aspects, the COF can be measured against silicone pads. In such exemplary and unlimiting aspects, the COF of the disclosed compounds remains low as compared to other polymers usually used to form the outer surface of the introducer. In some implementations, the COF remains low and without substantially a change over at least 10 cycles of friction testing.

In some implementations, wherein the compounds comprising polyurethane and PEO or the compounds comprising polyether block amide and PEO are used to form the outer surface of the introducer, the outer surface of the introducer demonstrates lubricious properties. It is understood that in such exemplary and unlimiting aspects, due to the lubricious properties of the outer surface, no additional hydrophilic coating is needed to be applied to the surface. In such aspects, the outer surface of the introducer does not comprise a hydrophilic coating.

In some implementations, where the compounds comprising polyurethane and PEO or the compounds comprising polyether block amide and PEO are used to form the outer surface of the introducer, such an introducer generates substantially no insoluble particulate material that can be released into a patient's body. This characteristic of the disclosed herein introducer is especially beneficial, as it further minimizes potential side effects, such as the release of nanoparticles into the patient's body that can accumulate in the patient's organs and affect recovery and longevity.

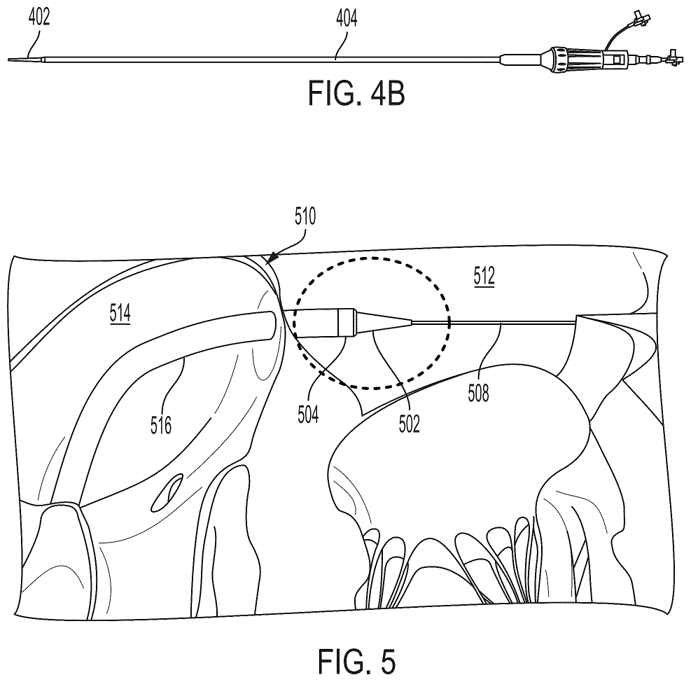

In some implementations, the proximal portion of the introducer 100 (FIG. 1A) is coupled with a hub 150 or an adaptor to connect with the medical device. In yet, the introducer 502 disclosed herein and as shown in FIG. 5 can be configured to receive a guidewire 508 used to deploy the medical device (not shown).

In some implementations, the sterilization process can be any known in the art sterilization process that can provide the desired level of sterilization for a medical device. In some aspects, the sterilization procedure is ethylene oxide-based. Ethylene oxide (also known as EO or EtO) is a low temperature gaseous process widely used to sterilize various medical devices. Ethylene oxide sterilization is a vacuum base. It is known that EO sterilization can efficiently penetrate the surfaces of most medical devices, and its lower temperature makes it an ideal process for a wide variety of materials. The ethylene oxide sterilization can be controlled by controlling gas concentration, humidity, temperature, and time. Generally, the humidity of the ethylene oxide gas used in the sterilization is up to about 70%, including exemplary values of about 10%, about 20%, about 30%, about 40%, about 50%, and about 60%.

Ethylene oxide is an alkylating agent. It can disrupt the cellular metabolism and reproductive processes of microorganisms resulting in eliminating of live microorganisms from the devices. Ethylene oxide can penetrate breathable packaging and various portions of the devices to allow contact with all accessible surfaces of the product to deliver the required sterility assurance level (SAL).

The introducers disclosed herein are designed to allow ethylene oxide gas, or other sterilizing fluid, for example, gaseous fluid, to travel and permeate through the narrow inner diameter of the introducer, with insubstantial, minimal or no ethylene oxide absorption in the introducer surfaces. For example, the presence of the hydrophobic second polymer layer allows ethylene oxide to travel through the lumen without substantially absorbing it within the second polymer layer.

The effect of sterilization can be assessed by a validation process to ensure the Sterility Assurance level. The validation is performed by placing a biological indicator (BI) within the introducer's location, where sterilizing conditions are the most difficult to achieve. In some exemplary and unlimiting aspects, the biological indicators can be placed at different locations within the narrow lumen to ensure that all lumen along the entire length of the introducer is sterilized to the desired level. Any known in the art biological indicators can be utilized. For example, and without limitations, the biological indicators can comprise Geobacillus or Bacillus species.

In some implementations, the introducers disclosed herein are configured to be substantially sterilized throughout of substantially entire d₁ and d₂ along substantially entire I₁, wherein substantially sterilized is defined by achieving effective inactivation of bio-indicators in a fractional cycle to less than about 1 CFU, less than about 0.9 CFU, less than about 0.8 CFU, less than about 0.7 CFU, less than about 0.6 CFU, less than about 0.5 CFU, less than about 0.4 CFU, less than about 0.3 CFU, less than about 0.2 CFU, less than about 0.1 CFU, less than about 0.09 CFU, less than about 0.08 CFU, less than about 0.07 CFU, less than about 0.06 CFU, less than about 0.05 CFU, less than about 0.04 CFU, less than about 0.03 CFU, less than about 0.02 CFU, or less than about 0.01 CFU.

In some implementations, the disclosed herein introducers can achieve a sterility assurance level of about 10-6 in the ethylene oxide sterilization.

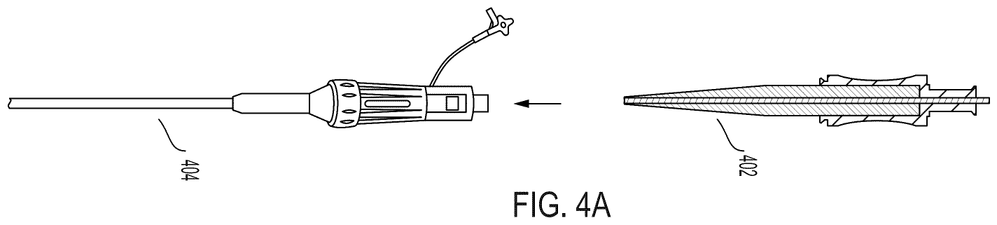

Also disclosed herein is a delivery system comprising: any of the disclosed herein introducers for the medical devices; a delivery sheath; and a prosthetic valve. FIG. 4A shows inserting an exemplary introducer 402 into a delivery sheath 404. FIG. 4B shows the introducer 402 inserted fully inserted into the delivery sheath 404, such that a tapered portion of the introducer is extended beyond a distal end of the delivery sheath.

It is understood that any known in the art delivery sheaths can be used. Some exemplary and unlimiting examples of the delivery sheath can be found in U.S. Pat. Nos. 9,987,134, 10,792,150, 10,524,905, 10,517,720, 10,254,906, 10,254,907, 11,045,317, 11,273,062, International Patent Application Publication No. PCT/US2021/019514, International Patent Application Publication No. PCT/US2021/050006, or Provisional Application Nos. 63/230,631 and 63/324,436, the contents of which are incorporated herein in their full entirety by reference.

In yet some implementations, the prosthetic valve can be a mitral valve.

### Methods

The aspects of the present disclosure also relate to methods of making the disclosed herein introducers for the medical devices, wherein the method comprises: forming an elongated tubing comprising a first polymer layer forming an outer surface of the introducer having an outer diameter d₁ and a second polymer layer; forming an inner surface of the introducer and defining a lumen having a diameter d₂, wherein the second layer is substantially more hydrophobic than the first layer; wherein the elongated tubing has a length I₁, wherein a ratio of I₁/d₁ is greater than about 120, and a ratio of h/d₂ is greater than about 200 to about 2,000; and wherein the introducer is configured to be substantially sterilized throughout of substantially entire d₁ and d₂ along substantially entire I₁.

In the aspects disclosed herein, the first polymer layer can comprise any of the disclosed above polymers. Still, in some implementations, the second polymer layer can comprise any of the disclosed above polymers.

It is understood that any known in the art method of forming elongated tubing can be utilized. In certain aspects, the elongated tubing can be extruded. In some aspects, each layer is extruded separately and then assembled together to form the desired configuration. Yet, in some implementations, the step of forming comprises co-extruding the first and the second polymer layers. In some implementations, the elongated tubing can further comprise a third polymer layer positioned between the first and the second polymer layers. In such aspects, the third layer can comprise any of the disclosed above polymers. In some implementations, the third polymer layer can be coextruded with the first and the second polymer layers. In certain exemplary aspects, the third polymer layer can be coextruded with the first polymer layer and then assembled with the second polymer layer. Yet, in some implementations, the third polymer layer can be coextruded with the second polymer layer and then assembled with the first polymer layer. In some implementations, all three polymer layers can be coextruded.

In some implementations, any or all of the polymer layers present in the introducer configuration can be at least partially crosslinked. In yet some implementations, any or all of the polymer layers present in the introducer configuration can be substantially crosslinked. In some implementations, crosslinking can be achieved by exposing any or all of the layers to an energy source, wherein the energy source can be selected from e-beam radiation, X-ray radiation, gamma-radiation, or electromagnetic radiation. In certain aspects, the crosslinking is achieved by exposure to the energy source at a dose of about 25 kGrey to about 100 kGrey, including exemplary values of about 30 kGrey, about 35 kGrey, about 40 kGrey, about 45 kGrey, about 50 kGrey, about 55 kGrey, about 60 kGrey, about 65 kGrey, about 70 kGrey, about 75 kGrey, about 80 kGrey, about 85 kGrey, about 90 kGrey, and about 95 kGrey.

Also disclosed herein are methods of delivering a prosthetic valve comprising: placing a guide wire; introducing any of the disclosed herein introducers; removing the guide wire; and placing a prosthetic valve in an implantation site. Exemplary procedural steps are shown in FIG. 5. The implantation site is prepped with an exemplary 23 Fr guide wire 508. The delivery sheath 516 penetrates the septum 510 and transfers the introducer 502 from a right atrium 514 to a left atrium 512. The introducer tip (or a distal tapered portion of the disclosed herein introducer) 502 can be seen extending from a guide sheath top 504.

It is understood that the above method(s) can be performed on a patient, living animal, or on a simulation, such as on a cadaver, cadaver heart, anthropomorphic ghost, simulator (e.g., with body parts, heart, tissue, etc. being simulated).

Also disclosed herein is a method comprising: exposing the disclosed herein introducer for the medical device to ethylene oxide gas comprising about 20% to about 70%, including exemplary values of about 25%, about 30%, about 35%, about 40%, about 45%, about 50%, about 55%, about 60%, and about 65% of humidity for a predetermined time at a predetermined temperature. It is understood that the predetermined time can be any time needed to achieve the desired level of sterilization. It is further understood that the level of sterilization can be assessed by placing a biological indicator (BI) within the introducer at a location where sterilizing conditions are the most difficult to achieve. In some exemplary and unlimiting aspects, the biological indicators can be placed at different locations within the narrow lumen to ensure that all lumen along the entire length of the introducer is sterilized to the desired level. Any known in the art biological indicators can be utilized. For example, and without limitations, the biological indicators can comprise Geobacillus or Bacillus species

In some implementations, the methods disclosed herein provide substantial sterilization throughout of substantially entire d₁ and d₂ along substantially entire I₁, wherein substantially sterilized is defined by achieving effective inactivation of bio-indicators in a fractional cycle to less than about 1 CFU, less than about 0.9 CFU, less than about 0.8 CFU, less than about 0.7 CFU, less than about 0.6 CFU, less than about 0.5 CFU, less than about 0.4 CFU, less than about 0.3 CFU, less than about 0.2 CFU, less than about 0.1 CFU, less than about 0.09 CFU, less than about 0.08 CFU, less than about 0.07 CFU, less than about 0.06 CFU, less than about 0.05 CFU, less than about 0.04 CFU, less than about 0.03 CFU, less than about 0.02 CFU, or less than about 0.01 CFU.

In yet some implementations, the disclosed herein introducers can achieve a sterility assurance level of about 10-6 in the ethylene oxide sterilization.

In some implementations, the step of exposing comprises one or more cycles of exposing to the ethylene oxide gas. In some implementations, the first polymer layer, the second polymer layer, and the third polymer layer, if present, show substantially no deterioration after the step of exposing.

### EXAMPLES

Disclosed herein is an introducer manufactured according to the aspects of the present disclosure. For example, disclosed herein is an introducer having I₁ of about 40.44±0.10 inch, d₁ of about 0.305±0.003 inch, and d₂ of about 0.039±0.001 inch, where the d₂ is substantially identical along the entire length I₁. A distal portion of the outer diameter is tapered. The tapered portion has a length I₂ of about 1.37±0.03 inches. The outer diameter at the most distal end of the introducer is about 0.065±0.003 inches.

The following table shows the sterilization results obtained for the inventive introducer as compared to a reference introducer. Control is a reference introducer having the disclosed above dimensions and is constructed from one polymer layer comprised of Shore 93A polyurethane, such as, for example, Tecoflex® 93A. Example 1 is an introducer having the disclosed above dimensions and having a coextruded polyurethane and High Density Polyethylene (HDPE) as the first and the second layers, respectively. In this example, the thickness of the first layer is 0.128″, and the thickness of the second layer is 0.005″. The first layer in this example has a composition of aliphatic polyether-based Tecoflex® 93A with 40 wt % Barium Sulfate. The second layer in this example has a composition of HDPE.

Example 2 is an introducer having the disclosed herein dimensions and having a first layer comprising a compound material of polyurethane and PEO and a second layer comprising HDPE, where both the first and the second layers are coextruded. In this example, the thickness of the first layer is 0.128″, and the thickness of the second layer is 0.005″. The first layer in this example has a composition of aromatic polyether-based polyurethane Pellethane® 55D with 30 wt % Poly(ethylene Oxide) and 20 wt % Barium Sulfate. The second layer in this example has a composition of HDPE.

D values are calculated per Stumbo Murphy Cochran where

$$D = \frac{U}{Log {N}_{0} - Log {N}_{u}} ,$$

wherein U is exposure time, N₀ is starting spore population, and Nᵤ is the most probable number of surviving spores. Nᵤ can be calculated:

Nᵤ=ln n/r, where n is a number of units exposed, and r is a number of units sterile (negative).

TABLE 1

| | | | | Total Inactivation | |
|---|---|---|---|---|---|
| | | Mean Long | Estimated | Time (minutes) | |
| | | Survivor Count/ | D Value | for worst-case | |
| Fractional | | 3Growth/# | (minutes) | Bioburden | |
| Cycle | Introducer | Samples | Estimate | reduction vs. BI | Interpretation |
| 60 min per | Control | 2.06 Max. 4.23 | 17.3 | [BI D value × | Not acceptable |
| cycle, EtO | | logs | | 4] = 69 min | |
| concentration | | | | | |
| of about 370 | | | | | |
| mg/L | | | | | |
| | Example 1 | ≤0.02 | ≤10.66 | [BI D value × | Acceptable |
| | | No survivors | | 4] = 42.6 min | |
| | Example 2 | 0.02 | 10.66 | [BI D value × | Acceptable |
| | | 1 survivors | | 4] = 42.6 min | |

### Exemplary Aspects

EXAMPLE 1: An introducer for a medical device comprising: an elongated tubing comprising a first polymer layer forming an outer surface of the introducer having an outer diameter d₁ and a second polymer layer forming an inner surface of the introducer and defining a lumen having a diameter d₂, wherein the second layer is substantially more hydrophobic than the first layer; wherein the elongated tubing has a length I₁; wherein a ratio of I₁/d₁ is greater than about 120 and a ratio of I₁/d₂ is greater than about 200 to about 2,000; and wherein the introducer is configured to be substantially sterilized throughout of substantially entire d₁ and d₂ along substantially entire I₁.

EXAMPLE 2: The introducer for the medical device of any examples herein, particularly example 1, wherein the second polymer layer comprises one or more polypropylene, polyethylene, a graft-modified polyethylene, or graft-modified polypropylene.

EXAMPLE 3: The introducer for the medical device of any examples herein, particularly example 2, wherein the second polymer layer comprises one or more of a low-density polyethylene (LDPE), medium density polyethylene, ultra-low-density polyethylene (ULDPE), high density polyethylene (HDPE), heterogeneously branched linear low-density polyethylene (LLDPE), a graft modified ethylene polymer, grafted modified polypropylene, or ethylene-vinyl acetate (EVA), or any combination thereof.

EXAMPLE 4: The introducer for the medical device of any examples herein, particularly example 2 or 3, wherein the graft-modified polyethylene or graft-modified polypropylene is maleic anhydride graft-modified or acrylic acid graft-modified.

EXAMPLE 5: The introducer for the medical device of any examples herein, particularly examples 1-4, wherein the first polymer layer comprises polyurethane, polyether block amide, polyethylene oxide, or any combination thereof.

EXAMPLE 6: The introducer for the medical device of any examples herein, particularly examples 1-5, wherein the first polymer layer is a thermoplastic polyurethane.

EXAMPLE 7: The introducer for the medical device of any examples herein, particularly examples 1-5, wherein the first polymer layer is a compound comprising greater than 0 wt % to less than 100 wt % of a polyether-based thermoplastic polyurethane and about 5 wt % to about 50 wt % of polyethylene oxide.

EXAMPLE 8: The introducer for the medical device of any examples herein, particularly examples 1-7, wherein the polyurethane has a durometer Shore D of about 50D to about 75D.

EXAMPLE 9: The introducer for the medical device of any examples herein, particularly examples 1-7, wherein the polyurethane has a Shore A of about 25A to about 95A.

EXAMPLE 10: The introducer for the medical device of any examples herein, particularly examples 1-5, wherein the first polymer layer is a polyether block amide.

EXAMPLE 11: The introducer for the medical device of any examples herein, particularly examples 1-5, wherein the first polymer layer is a compound comprising greater than 0 wt % to less than 100 wt % of a polyether block amide and about 5 wt % to about 50 wt % of polyethylene oxide.

EXAMPLE 12: The introducer for the medical device of any examples herein, particularly examples 1-5 or 10-11, wherein the polyether block amide has a Shore hardness of about 25 D to about 72 D.

EXAMPLE 13: The introducer for the medical device of any examples herein, particularly examples 7-9 or 11-12, wherein the polyethylene oxide (PEO) has a molecular weight of about 100,000 to about 7,000,000.

EXAMPLE 14: The introducer for the medical device of any examples herein, particularly examples 1-13, wherein the first polymer layer has a thickness of about 0.005 inches to about 0.150 inches.

EXAMPLE 15: The introducer for the medical device of any examples herein, particularly examples 1-14, wherein the second polymer layer has a thickness from about 0.005 inches to about 0.035 inches.

EXAMPLE 16: The introducer for the medical device of any examples herein, particularly examples 1-15, further comprises a third polymer layer positioned between the first and the second polymer layers.

EXAMPLE 17: The introducer for the medical device of any examples herein, particularly example 16, wherein the third polymer layer is a tie layer and comprises a graft-modified polyethylene or graft-modified polypropylene.

EXAMPLE 18: The introducer for the medical device of any examples herein, particularly examples 16 or 17, wherein the third polymer layer has a thickness of about 0.001 inches to about 0.025 inches.

EXAMPLE 19: The introducer for the medical device of any examples herein, particularly examples 1-18, wherein the first polymer layer and/or the second polymer layer comprise a filler.

EXAMPLE 20: The introducer for the medical device of any examples herein, particularly examples 16-19, wherein the third polymer layer comprises a filler.

EXAMPLE 21: The introducer for the medical device of any examples herein, particularly example 20, wherein the filler comprises bismuth oxychloride, barium sulfate, bismuth subcarbonate, calcium carbonate, aluminum trihydrate, barite, kaolin clay, limestone, titanium oxide, or any combination thereof.

EXAMPLE 22: The introducer for the medical device of any examples herein, particularly examples 1-21, wherein the first and the second polymer layers are co-extruded.

EXAMPLE 23: The introducer for the medical device of any examples herein, particularly examples 16-22, wherein the first polymer layer and/or the second polymer layer and/or third layer are co-extruded.

EXAMPLE 24: The introducer for the medical device of any examples herein, particularly examples 1-23, wherein the first polymer layer and/or the second polymer layer are at least partially crosslinked.

EXAMPLE 25: The introducer for the medical device of any examples herein, particularly example 24, wherein the crosslinking is achieved by exposure to an energy source, wherein the energy source is selected from e-beam radiation, X-ray radiation, gamma-radiation, or electromagnetic radiation.

EXAMPLE 26: The introducer for the medical device of any examples herein, particularly example 25, wherein the crosslinking is achieved by exposure to the energy source at a dose of about 25 kGrey to about 100 kGrey.

EXAMPLE 27: The introducer for the medical device of any examples herein, particularly examples 1-26, wherein the first polymer layer and/or the second polymer layer are substantially crosslinked.

EXAMPLE 28: The introducer for the medical device of any examples herein, particularly examples 1-27, wherein the inner diameter d₂ is substantially the same along the entire length I₁.

EXAMPLE 29: The introducer for the medical device of any examples herein, particularly examples 1-28, wherein the introducer has a proximal portion and a distal portion.

EXAMPLE 30: The introducer for the medical device of any examples herein, particularly example 29, wherein the proximal portion is coupled with a hub or an adaptor to connect a medical device.

EXAMPLE 31: The introducer for the medical device of any examples herein, particularly examples 29 or 30, wherein the distal portion is tapered such that the outer diameter d₁ decreases to a distal end of the introducer while the inner diameter di remains substantially the same along the entire length I₁.

EXAMPLE 32: The introducer for the medical device of any examples herein, particularly examples 1-31, wherein the introducer is configured to receive a guidewire used to deploy the medical device.

EXAMPLE 33: The introducer for the medical device of any examples herein, particularly examples 1-32, wherein the sterilization is ethylene oxide-based.

EXAMPLE 34: The introducer for the medical device of any examples herein, particularly examples 1-33, wherein substantially sterilized throughout of substantially entire d₂ along substantially entire I₁ defined by achieving effective inactivation of bio-indicators in a fractional cycle to less than about 1 CFU.

EXAMPLE 35: The introducer for the medical device of any examples herein, particularly example 34, wherein a sterility assurance level is about 10-6.

EXAMPLE 36: The introducer for the medical device of any examples herein, particularly examples 7-9 or 11-35, wherein the outer surface of the introducer is substantially lubricious and wherein exhibits a Coefficient of Friction (COF) of about 0.05 to about 0.5.

EXAMPLE 37: The introducer for the medical device of any examples herein, particularly examples 7-9 or 11-36, wherein the outer surface of the introducer does not comprise a hydrophilic coating.

EXAMPLE 38: The introducer for the medical device of any examples herein, particularly examples 7-9 or 11-37, wherein the introducer is configured to generate substantially no insoluble particulate material.

EXAMPLE 39: A delivery system comprising: the introducer for the medical device of any examples herein, particularly examples 1-38; a delivery sheath; and a prosthetic valve.

EXAMPLE 40: A method of making the introducer for the medical device of any examples herein, particularly examples 1-38 comprising: forming an elongated tubing comprising a first polymer layer forming an outer surface of the introducer having an outer diameter d₁ and a second polymer layer; forming an inner surface of the introducer and defining a lumen having a diameter d₂, wherein the second layer is substantially more hydrophobic than the first layer; wherein the elongated tubing has a length I₁, wherein a ratio of I₁/d₁ is greater than about 120 and a ratio of I₁/d₂ is greater than about 200 to about 2,000; and wherein the introducer is configured to be substantially sterilized throughout of substantially entire d₁ and d₂ along substantially entire I₁.

EXAMPLE 41: The method of any examples herein, particularly example 40, wherein the step of forming comprises co-extruding the first and the second polymer layers.

EXAMPLE 42: The method of any examples herein, particularly example 40 or 41, wherein the elongated tubing further comprises a third polymer layer positioned between the first and the second polymer layers.

EXAMPLE 43: The method of any examples herein, particularly example 42, wherein the third polymer layer is coextruded with the first and the second polymer layers.

EXAMPLE 44: A method of delivering a prosthetic valve comprising: placing a guide wire; introducing the introducer of any one of examples herein, particularly examples 1-38, removing the guide wire; and placing a prosthetic valve in an implantation site.

EXAMPLE 45: A method comprising: exposing the introducer for the medical device any examples herein, particularly examples 1-38, to ethylene oxide gas comprising about 20% to about 70% of humidity for a predetermined time at a predetermined temperature.

EXAMPLE 46: The method of any examples herein, particularly example 45, wherein the step of exposing comprises one or more cycles of exposing to the ethylene oxide gas.

EXAMPLE 47: The method of any examples herein, particularly example 45 or 46, wherein the step of exposing achieves effective inactivation of bio-indicators in a fractional cycle to less than about 1 CFU.

EXAMPLE 48: The method of any examples herein, particularly example 47, wherein the step of exposing achieves a sterility assurance level is about 10-6.

EXAMPLE 49: The method of any examples herein, particularly examples 45-48, wherein the first polymer layer, the second polymer layer, and the third polymer layer show substantially no deterioration after the step of exposing.

In view of the many possible aspects to which the principles of the disclosed disclosure can be applied, it should be recognized that the illustrated aspects are only some examples of the disclosure and should not be taken as limiting the scope of the disclosure. Rather, the scope of the disclosure is defined by the following claims. We, therefore, claim as our disclosure all that comes within the scope and spirit of these claims.

We claim:

1. An introducer for a medical device, the introducer comprising:
an elongated tubing comprising a first polymer layer forming an outer surface of the introducer having an outer diameter d₁ and a second polymer layer forming an inner surface of the introducer and defining a lumen having a diameter d₂, wherein the second layer is substantially more hydrophobic than the first layer;
wherein the elongated tubing has a length I₁,
wherein a ratio of I₁/d₁ is greater than about 120 and a ratio of I₁/d₂ is greater than about 200 to about 2,000;
and wherein the introducer is configured to be substantially sterilized throughout of substantially entire d₁ and d₂ along substantially entire I₁.

2. The introducer of claim 1, wherein the second polymer layer comprises one or more polypropylene, polyethylene, a graft-modified polyethylene, or graft-modified polypropylene.

3. The introducer of claim 2, wherein the second polymer layer comprises one or more of a low-density polyethylene (LDPE), medium density polyethylene, ultra-low-density polyethylene (ULDPE), high density polyethylene (HDPE), heterogeneously branched linear low-density polyethylene (LLDPE), a graft modified ethylene polymer, grafted modified polypropylene, or ethylene-vinyl acetate (EVA), or any combination thereof.

4. The introducer of claim 1, wherein the first polymer layer comprises polyurethane, polyether block amide, polyethylene oxide, or any combination thereof.

5. The introducer of claim 1, wherein the first polymer layer is a compound comprising greater than 0 wt % to less than 100 wt % of a polyether-based thermoplastic polyurethane and about 5 wt % to about 50 wt % of polyethylene oxide.

6. The introducer of claim 4, wherein the polyurethane has a durometer Shore D of about 50D to about 75D and/or a Shore A of about 25A to about 95A.

7. The introducer of claim 1, wherein the first polymer layer is a compound comprising greater than 0 wt % to less than 100 wt % of a polyether block amide and about 5 wt % to about 50 wt % of polyethylene oxide.

8. The introducer of claim 7, wherein the polyether block amide has a Shore hardness of about 25 D to about 72 D.

9. The introducer of claim 7, wherein the polyethylene oxide (PEO) has a molecular weight of about 100,000 Daltons to about 7,000,000 Daltons.

10. The introducer of claim 1, wherein the first polymer layer has a thickness of about 0.005 inches to about 0.150 inches and/or wherein the second polymer layer has a thickness of about 0.005 inches to about 0.035 inches.

11. The introducer of claim 1, wherein the introducer has a proximal portion and a distal portion, wherein the proximal portion is coupled with a hub or an adaptor to connect with the medical device, and wherein the distal portion is tapered such that the outer diameter d₁ decreases to a distal end of the introducer while the inner diameter d₁ remains substantially the same along the entire length I₁.

12. The introducer of claim 1, wherein the introducer is configured to receive a guidewire used to deploy the medical device.

13. The introducer of claim 1, wherein the sterilization is ethylene oxide-based.

14. The introducer of claim 1, wherein substantially sterilized throughout of substantially entire d₂ along substantially entire I₁ defined by achieving effective inactivation of bio-indicators in a fractional cycle to less than about 1 CFU and/or wherein a sterility assurance level is about 10-6.

15. The introducer of claim 5, wherein the outer surface of the introducer is substantially lubricious and wherein exhibits a Coefficient of Friction (COF) of about 0.05 to about 0.5, and wherein the outer surface of the introducer does not comprise a hydrophilic coating.

16. A method of making an introducer for a medical device, the method comprising:
forming an elongated tubing comprising a first polymer layer forming an outer surface of the introducer having an outer diameter d₁ and a second polymer layer;
forming an inner surface of the introducer and defining a lumen having a diameter d₂, wherein the second layer is substantially more hydrophobic than the first layer;
wherein the elongated tubing has a length I₁,
wherein a ratio of I₁/d₁ is greater than about 120 and a ratio of I₁/d₂ is greater than about 200 to about 2,000;
and wherein the introducer is configured to be substantially sterilized throughout of substantially entire d₁ and d₂ along substantially entire I₁.

17. The method of claim 16, wherein the step of forming comprises co-extruding the first and the second polymer layers.
